(19) United States
(12) Patent Application Publication
(10) Pub. No.: US 2018/0049650 A1
(43) Pub. Date: Feb. 22, 2018

(54) BLOOD PARAMETER MONITORING DEVICE AND BLOOD PARAMETER MONITORING METHOD

(71) Applicant: BOE TECHNOLOGY GROUP CO., LTD., Beijing (CN)

(72) Inventors: Hong ZHANG, Beijing (CN); Xuewen LV, Beijing (CN); Yan LU, Beijing (CN)

(73) Assignee: BOE TECHNOLOGY GROUP CO., LTD., Beijing (CN)

(21) Appl. No.: 15/554,174

(22) PCT Filed: Apr. 21, 2016
(86) PCT No.: PCT/CN2016/079831

§ 371 Date: Aug. 28, 2017

(30) Foreign Application Priority Data
May 14, 2015 (CN) 201510246079.0

(51) Int. Cl. A61B 5/0205; A61B 5/00

(52) U.S. Cl. CPC A61B 5/0205; A61B 5/6833; A61B 5/026; A61B 5/0022; A61B 5/746

(57) ABSTRACT

Provided is a blood parameter monitoring device, including: a skin patch wearable on skin; a blood parameter sensor arranged at the skin patch and configured to detect a blood parameter; a processor configured to send an abnormality indication signal when determining that the blood parameter is not within a predetermined range, and send a normality indication signal when determining that the blood parameter is within the predetermined range; a timer configured to start a timing process upon receiving the abnormality indication signal, stop the timing process upon receiving the normality indication signal, and send an alarm signal if a duration of the timing process is longer than or equal to a predetermined duration; and an alarm connected to the timer and configured to give an alarm and/or trigger a predetermined mobile communication terminal to send a warning signal to a preset phone number, after receiving the alarm signal.

### CROSS-REFERENCE TO RELATED APPLICATION

The present application claims priority to Chinese Patent Application No. 201510246079.0 filed on May 14, 2015, the disclosure of which is incorporated in its entirety by reference herein.

### TECHNICAL FIELD

The present disclosure relates to the technical field of biochemical micro-fluidic detection, and in particular to a blood parameter monitoring device and a blood parameter monitoring method.

### BACKGROUND

At present, population aging becomes a worldwide issue, and China is entering the stage of population aging. Chronic disease is an important factor affecting the health of the elderly since it may cause local lesion, local necrosis or the like at an organ. At the initial stage of the chronic disease, abnormality may occur at only a part, in particular, at an extremity such as a finger or toe. Such initial symptom of the chronic disease is generally not obvious and thus is not easy to be perceived and consequently cannot be treated timely. Later more parts may get diseased, the symptom of the chronic disease becomes obvious and easily perceived; however, the chronic disease is then serious and best treatment time is missed, the disease cannot be cured and the life cannot be rescued.

The old man is prone to suffering from diseases such as acute cerebral infarction or cerebral hemorrhage. People having a family history of these diseases are more risky to suffer from these diseases, and the life is greatly threatened if these diseases cannot be detected and treated timely.

In addition, some special groups such as children cannot express their discomfort accurately, and thus their discomfort is prone to being omitted or cannot be detected timely. In one case, a child is injured at his/her head or other parts since he/she fell when playing, parents of the child may consider that it is unnecessary to go to hospital to check the body, however, the head or other parts of the child may have serious problems due to the injury, for example, local chronic bleeding in head, which may cause a permanent damage in brain if it cannot be treated timely. In another case, when the child is sleeping, he/she cannot turn over since he/she is too young, the limbs of the child may be numb, and long time numb of the limbs may cause a lesion of an organ or even suffocation.

In view of the above, it is desired a wearable device capable of detecting a local lesion of an organ timely and conveniently and a method for detecting a local lesion of an organ.

### SUMMARY

A blood parameter monitoring device and a blood parameter monitoring method are provided according to the present disclosure, to solve a problem in related technology that it is difficult to provide a wearable device that can detect a local lesion of an organ timely.

A blood parameter monitoring device is provided according to the present disclosure. The device includes:

a skin patch, which is wearable on skin of an organism;

a blood parameter sensor, which is arranged at the skin patch and configured to detect a blood parameter of the organism;

a processor, which is connected to the blood parameter sensor and configured to send an abnormality indication signal in case of determining that the blood parameter detected by the blood parameter sensor is not within a predetermined range, and send a normality indication signal in case of determining that the blood parameter detected by the blood parameter sensor is within the predetermined range;

a timer, which is connected to the processor and configured to start a timing process upon receiving the abnormality indication signal, stop the timing process upon receiving the normality indication signal, and send an alarm signal in a case that a duration of the timing process is longer than or equal to a predetermined duration; and

an alarm, which is connected to the timer and configured to give an alarm after receiving the alarm signal.

Optionally, the blood parameter monitoring device further includes a predetermined mobile communication terminal, and the alarm may be further configured to trigger, after receiving the alarm signal, the predetermined mobile communication terminal to send a warning signal to a preset phone number.

Optionally, the alarm includes a transmitting element configured to transmit triggering information to the predetermined mobile communication terminal after the alarm signal is received, to trigger the predetermined mobile communication terminal to dial the preset phone number and/or send a warning message to the preset phone number.

Optionally, the blood parameter sensor includes at least one of a blood flow sensor, a blood pressure sensor, a blood vessel resistance detector and a blood oxygen sensor.

Optionally, the processor, the timer and the alarm are all arranged at the skin patch.

Optionally, the processor, the timer and the alarm are all integrated in the predetermined mobile communication terminal. The blood parameter sensor further includes: a first communication element arranged at the skin patch and configured to transmit the blood parameter to the processor. A second communication element communicatively connected to the first communication element is arranged in the predetermined mobile communication terminal. The predetermined mobile communication terminal receives the blood parameter and transfers the blood parameter to the processor, via the second communication element.

Optionally, the predetermined mobile communication terminal may be a phone, a computer or other device having a communication function. The first communication element and the second communication element may be Bluetooth communication elements, infrared communication element or other wireless communication elements.

Optionally, the skin patch is a flexible patch.

Optionally, the flexible patch is made of a plastic or a rubber, and has a strip shape. The flexible patch having the strip shape has one end provided with at least one circular hole, and another end provided with a convex matching each of the at least one circular hole. Or, the flexible patch having the strip shape has two ends each provided with an adhesive portion.

Optionally, the flexible patch is made of a synthetic fiber or a natural fiber. The flexible patch is provided with a fixing portion that enables the flexible patch to be wearable on the skin of the organism.

Optionally, the flexible patch includes an electro-active fabric, and the blood parameter sensor and the flexible patch are integral.

Optionally, in a case that the processor, the timer and the alarm are all arranged at the skin patch, the blood parameter sensor further includes a power source configured to provide electric energy to the processor, the timer and the alarm, and the power source is directly integrated in the flexible patch.

A blood parameter monitoring method is further provided according to the present disclosure. The method is applied in a blood parameter monitoring device. The blood parameter monitoring device includes a skin patch, a blood parameter sensor arranged at the skin patch, a processor connected to the blood parameter sensor, a timer connected to the processor and an alarm connected to the timer. The blood parameter monitoring method includes:

wearing the skin patch on skin of an organism;

detecting, by the blood parameter sensor, a blood parameter of the organism;

determining, by the processor, whether the blood parameter detected by the blood parameter sensor is within a predetermined range, sending an abnormality indication signal in a case that the blood parameter is not within the predetermined range, and sending a normality indication signal in a case that the blood parameter is within the predetermined range; and

starting, by the timer, a timing process upon receiving the abnormality indication signal, stopping, by the timer, the timing process upon receiving the normality indication signal, and sending, by the timer, an alarm signal in a case that a duration of the timing process is longer than or equal to a predetermined duration.

Optionally, the blood parameter monitoring device further includes a predetermined mobile communication terminal, and the blood parameter monitoring method further includes:

triggering, by the alarm after receiving the alarm signal, the predetermined mobile communication terminal to send a warning signal to a preset phone number.

In the blood parameter monitoring device and blood parameter monitoring method according to the present disclosure, the blood parameter sensor is arranged at the skin patch wearable on skin of an organism. An alarm may be given if a duration in which a blood parameter is abnormal reaches a predetermined duration, and the alarm may be given in a manner of direct sound-light alarm and/or by dialing a preset phone number or sending a message to the preset phone number. An alarm signal may be sent if it is detected that the blood parameter of the organism is in an abnormal state for a long time, to remind related person to timely do a medical examination to prevent risking his life.

### BRIEF DESCRIPTION OF THE DRAWINGS

FIG. 1 is a structural diagram of a blood parameter monitoring device according to some embodiments of the present disclosure;

FIG. 2 is a schematic structural diagram of a blood parameter monitoring device according to some embodiments of the present disclosure;

FIG. 3 is a schematic structural diagram of a blood parameter monitoring device according to some embodiments of the present disclosure;

FIG. 4 is a structural diagram of components of the blood parameter monitoring device shown in FIG. 3 that are arranged at a mobile terminal; and

FIG. 5 is a flowchart of a blood parameter monitoring method according to some embodiments of the present disclosure.

### DETAILED DESCRIPTION OF THE EMBODIMENTS

Hereinafter, the technical solution in the embodiments of the present disclosure will be described clearly and completely in conjunction with the drawings in the embodiments of the present disclosure. Obviously, the described embodiments are only a part of the embodiments of the present disclosure, but not all the embodiments. All the other embodiments obtained by those skilled in the art based on the embodiments of the present disclosure without any creative work fall within the scope of protection of the present disclosure.

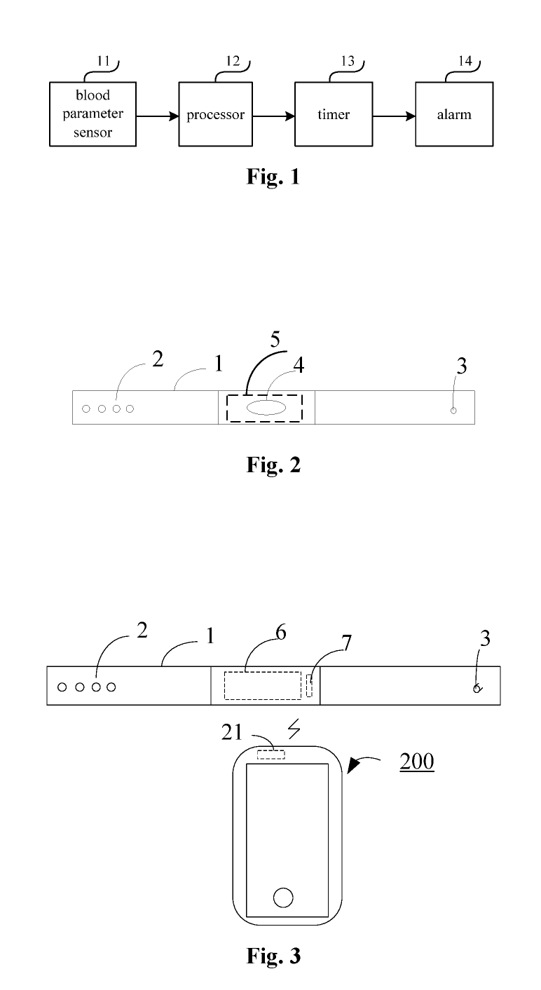

As shown in FIG. 1, a blood parameter monitoring device according to some embodiments of the present disclosure includes a skin patch (not shown in FIG. 1), a blood parameter sensor 11, a processor 12, a timer 13 and an alarm 14.

The skin patch is wearable on skin of an organism.

The blood parameter sensor 11 is arranged at the skin patch and configured to detect a blood parameter of the organism.

The processor 12 is connected to the blood parameter sensor 11, and is configured to send an abnormality indication signal in case of determining that that the blood parameter detected by the blood parameter sensor 11 is not within a predetermined range, and send a normality indication signal in case of determining that the blood parameter detected by the blood parameter sensor 11 is within the predetermined range. The processor 12 and the blood parameter sensor 11 may be directly connected to each other via a wire or may be communicatively connected to each other.

The timer 13 is connected to the processor 12, and is configured to start a timing process upon receiving the abnormality indication signal, stop the timing process upon receiving the normality indication signal, and send an alarm signal in the case that a duration of the timing process is longer than or equal to a predetermined duration.

The alarm 14 is connected to the timer 13 and configured to give an alarm after receiving the alarm signal.

Optionally, the blood parameter monitoring device further includes a predetermined mobile communication terminal, and the alarm may be further configured to trigger, after receiving the alarm signal, the predetermined mobile communication terminal to send a warning signal to a preset phone number.

In the blood parameter monitoring device according to the embodiments of the present disclosure, the blood parameter sensor is arranged at the skin patch wearable on skin of an organism. An alarm may be given if a duration in which a blood parameter is abnormal reaches a predetermined duration, and the alarm may be given in a manner of direct sound-light alarm and/or by dialing a preset phone number or sending a message to the preset phone number. An alarm signal may be sent if it is detected that the blood parameter of the organism is in an abnormal state for a long time, to remind related person to timely do a medical examination to prevent risking his life.

In the case that the alarm needs to directly give an alarm after receiving the alarm signal, the alarm may give the alarm by giving a warning sound or by vibrating, by displaying or glowing, or in other manner capable of warning the organism or related person wearing the skin patch.

In the case that the alarm needs to trigger, after receiving the alarm signal, the predetermined mobile communication terminal to send the warning signal to the preset phone number, the alarm may include a transmitting element configured to transmit triggering information to the predetermined mobile communication terminal after the alarm signal is received, to trigger the predetermined mobile communication terminal to dial the preset phone number and/or send a warning message to the preset phone number. For example, if a blood parameter of the elderly or baby at home is in an abnormal state for a long time, this situation may be reported to a related person working outside by a call or a message; hence, the elderly or baby may be medically treated timely.

The blood parameter sensor may include at least one of a blood flow sensor, a blood pressure sensor, a blood vessel resistance detector and a blood oxygen sensor. In an actual operation, the blood parameter sensor may also include any other components capable of detecting other parameters associated with blood, which are not described herein.

According to an embodiment, the processor, the timer and the alarm may all be arranged at the skin patch.

According to an embodiment, the processor, the timer and the alarm may all be integrated in the predetermined mobile communication terminal. The blood parameter sensor further includes a first communication element arranged at the skin patch and configured to transmit the blood parameter to the processor. The predetermined mobile communication terminal includes a second communication element communicatively connected to the first communication element. The predetermined mobile communication terminal receives the blood parameter and transfers the blood parameter to the processor, via the second communication element.

The predetermined mobile communication terminal may be a phone, a computer or other devices having a communication function. The first communication element and the second communication element may be wireless communication elements, such as Bluetooth communication elements, infrared communication elements or other wireless communication elements. In the above embodiments, the second communication element may be a module which is originally arranged in the predetermined mobile communication terminal, a module having a determining function may be added into a main chip of the predetermined mobile communication terminal, and the alarm may be implemented by a display and/or a loudspeaker of the predetermined mobile communication terminal. In this case, the solution can be achieved conveniently since only a functional module needs to be added in the main chip without adding hardware to the predetermined mobile communication terminal. Therefore, the solution is adapted to any mobile terminal having a wireless communication function.

Optionally, the skin patch may be a flexible patch. In this case, the skin patch can fit with the skin of the organism, thereby detecting the blood parameter more accurately.

The flexible patch may be made of plastic or rubber and may have a strip shape. The strip-shaped flexible patch has one end provided with at least one circular hole, and the other end provided with a convex matching the at least one circular hole. Or, the flexible patch has two ends each provided with an adhesive portion. In this way, it is convenient to wear the flexible patch on a part of the organism, such as finger, wrist or foot.

The flexible patch may be made of synthetic fibers or natural fibers, and the flexible patch is provided with a fixing portion, such that the flexible patch can be worn on the skin of the organism. The organism may fell much more comfortable when wearing the flexible patch made of synthetic fibers or natural fibers.

Optionally, the flexible patch may include an electro-active fabric, such that the blood parameter sensor and the flexible patch are integral.

Optionally, in the case that the processor, the timer and the alarm are arranged at the skin patch, the blood parameter sensor may further include a power source configured to provide electric energy to the processor, the timer and the alarm, and the power source is directly integrated within the flexible patch.

In the above preferable embodiments of the present disclosure, a conductive circuit is formed in the fabric of the flexible patch. A component which can be integrated with the flexible patch includes but is not limited to: a power source, a processor, a communication element, a sensor and other circuit elements. These components may be embedded in the flexible patch, knitted in the flexible patch or attached to an inner surface of the flexible patch instead of being installed on an outer surface of the flexible patch. Alternatively, the components can be installed on an outer surface of the flexible patch.

The flexible patch may be made of polyester fibers, nylon fibers, polyurethane fibers or other synthetic fibers or natural fibers. In an embodiment, the patch is elastic which is due to characteristic of the fibers or is due to the process of forming the patch using the fibers. The flexible patch may be woven, nonwoven, knitted, silk-spun or made of a woven film, and the flexible patch is preferably made of an electro-active fabric. Electro-conductivity of the fabric may be achieved with fine metal wires. The metal wires may be included in yarns of the fabric for forming the patch or may be woven into the fabric along with normal fabric fibers. Alternatively, the electro-conductivity of the flexible patch may be due to an inherent conductive polymer or nano-composite depositing on the fabric fibers as a coating.

Optionally, the flexible patch may be soft, elastic and air permeable, thereby increasing the comfortable sensation of a user using the flexible patch. The fabric of the flexible patch may be curled, wrinkled and folded without affecting its functionality. The flexible patch may be constituted or coated to be inflaming retarding, water resistant or waterproof.

The blood parameter monitoring device according to the embodiments of the disclosure includes a flexible patch. In an actual operation, the flexible patch is worn on hand, foot or other parts having a disease risk. A sensor is arranged in the flexible patch, for detecting a blood parameter. The blood parameter, for example, may include but is not limited to at least one of a blood flow, a blood pressure and a blood resistance. In the case that the blood parameter is in an abnormal state for a duration exceeding a predetermined duration, an alarm may be given in a manner of ringing, vibrating, glowing, displaying, dialing a number, sending a warning message or the like, to remind a related person to do a medical examination timely to prevent risking his life.

An actual operation of the blood parameter monitoring device according to the embodiments of the present disclosure includes steps S1 to S3.

In step S1, the flexible patch is worn on a part of a human body, such as a finger, a wrist or a foot.

The flexible patch may be made of plastic, rubber or other flexible material. The flexible patch may be worn on a part to be monitored, that is, a position of an organ prone to being diseased. For example, the flexible patch may be used to wrap the finger, and of course, may be worn on the foot or head. The flexible patch may have a strip shape and is flexible; hence it may be used to wrap a part such as finger, toe, head or the like.

In step S2, a real-time monitoring is performed with the flexible patch.

The flexible patch has a blood parameter sensor arranged therein. The blood parameter sensor, for example, may include at least one of a blood flow sensor, a blood pressure sensor, a blood vessel resistance detector and a blood oxygen sensor. In the case that a skin surface of an organ part to be monitored is wrapped with the flexible patch, parameters including a blood flow, a blood pressure, a blood resistance, a blood oxygen and the like of blood flowing through the organ part can be detected. If the monitored organ part is diseased, blood parameters such as the blood flow, the blood pressure, the blood resistance or the blood oxygen around the organ part may be changed. For example, if an organ part is necrotic, the blood flow corresponding to the organ is apparently decreased. A blood flow in brain may be decreased in case of a cerebral infarction, and thus a blood pressure in brain may be decreased. The blood flow in brain may be increased in case of a cerebral hemorrhage, and thus the blood pressure may be increased. If a limb is numb, a blood flow corresponding to the limb may be decreased. In the case that the blood parameter corresponding to the organ part is changed, the blood parameter sensor can detect a current blood parameter corresponding to the organ part, and transfer the detected blood parameter to a processor.

In step S3, it is determined whether a potential risk exists.

The processor sends an abnormality indication signal after it determines that the blood parameter is not within a predetermined range, and sends a normality indication signal after it determines that the blood parameter is within the predetermined range. The timer starts a timing process upon receiving the abnormality indication signal, stops the timing process upon receiving the normality indication signal, and sends an alarm signal if a duration of the timing process is longer than or equal to a predetermined duration. After receiving the alarm signal, the alarm may give an alarm and/or trigger the predetermined mobile communication terminal to send a warning signal to a preset phone number.

After receiving the alarm signal, the alarm may give an alarm by sounding, outputting an audio, vibrating, displaying, triggering the predetermined mobile communication terminal to dial the preset phone number or triggering the predetermined mobile communication terminal to send a warning message to the preset phone number, to remind a user of a current risk for reference. Of course, this reminder only indicates that the user currently has a potential risk, and the user may further confirm by doing an examination in hospital. It is also possible that the user is now in a dangerous situation; in this case, the reminder can remind the user and his/her family and the user can be medically treated in time.

In the following, a blood parameter monitoring device according to the present disclosure is described according to embodiments.

A blood parameter monitoring device according to some embodiments of the present disclosure has a structure as shown in FIG. 2. The blood parameter monitoring device includes a flexible patch 1 having a strip shape. The flexible patch 1 is made of plastic. The flexible patch has one end provided with several circular holes 2 and the other end provided with a convex 3 matching the circular holes 2. The flexible patch 1 may be worn on a part of human body to be monitored in a surrounding way. For example, the flexible patch may be worn on finger, toe, wrist, head or the like, and the convex 3 may be snapped into a corresponding circular hole 2. An appropriate circular hole 2 can be selected among the several circular holes 2 based on the size of a part to wear the flexible patch and the convex 3 is snapped into the selected circular hole 2, such that the flexible patch 1 can be tightly attached to the part to wear the flexible patch. Of course, the flexible patch 1 may have other shapes, as long as it can be suitably worn on a part of human body to be monitored.

In the blood parameter monitoring device according to the above embodiments, a blood parameter sensor, a processor, a timer and an alarm 4 may be arranged in the flexible patch 1. In addition, the alarm 4 may be a flickering LED lamp, or a loudspeaker capable of sounding, which is not limited herein.

In FIG. 2, a rectangular dashed box 5 illustratively identifies a functional module including the blood parameter sensor, the processor and the timer.

A structure of a blood parameter monitoring device according to some embodiments of the present disclosure is shown in FIG. 3. The structure of the blood parameter monitoring device is similar to that of the blood parameter monitoring device in FIG. 2, and is different from that of the blood parameter monitoring device in FIG. 2 in that: no processor, timer or alarm are arranged at a flexible patch 1 included in the blood parameter monitoring device. The flexible patch 1 included in the blood parameter monitoring device has a strip shape, and of course, may have other shapes, as long as it can be suitably worn on a part of human body to be monitored. The flexible patch 1 is made of plastic material. The flexible patch 1 has one end provided with several circular holes 2, and the other end provided with a convex 3 matching the circular holes 2. The flexible patch 1 may be worn on a to-be-monitored part of human body in a surrounding way. For example, the flexible patch 1 may be worn on finger, toe, wrist, head or the like by snapping the convex 3 may into a corresponding circular hole 2. An appropriate circular hole 2 may be selected among the several circular holes 2 based on the size of a part to wear the flexible patch 1, and the convex 3 is snapped into the selected circular hole 2. In this case, the flexible patch 1 can be tightly attached to the part to wear the flexible patch 1.

In the flexible patch 1, a blood parameter sensor 6 is arranged, and a function corresponding to the processor and a function corresponding to the alarm can be achieved by a mobile terminal capable of communicating with the flexible patch 1.

As shown in FIG. 3, a first wireless communication element 7 is arranged at the flexible patch 1. The first wireless communication element 7, for example, may be a Bluetooth communication element or an infrared communication element. The blood parameter sensor 6 transmits a detected blood parameter to a mobile terminal 200 via the first wireless communication element 7. The mobile terminal 200 may be a phone, a computer or other devices having a communication function. The mobile terminal 200 may receive the blood parameter via a second wireless communication element 21 in the mobile terminal 200. In this case, a wireless communication between the mobile terminal 200 and the flexible patch 1 is achieved.

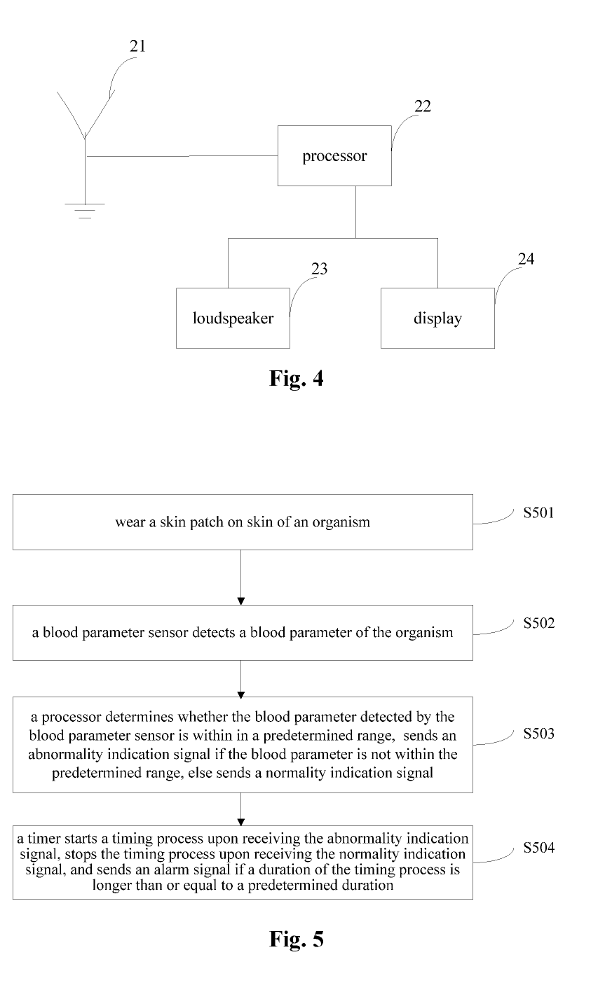

As shown in FIG. 4, a processor 22 having a determining function may be added in the mobile terminal. After receiving the blood parameter, the second wireless communication element 21 of the mobile terminal transmits the blood parameter to the processor 22 of the mobile terminal. The processor 22 performs a determination. If the detected blood parameter exceeds a threshold, or if the detected blood parameter is lower than a certain threshold, or if the detected parameter exceeds the threshold for a certain duration, or if the detected parameter is lower than a certain threshold for a certain duration, an alarm signal is output via a loudspeaker 23 of the mobile terminal or is displayed by a display screen 24 of the mobile terminal. In this case, the loudspeaker 23 and display screen 24 of the mobile terminal can serve as the alarm.

In the blood parameter monitoring device according to the above embodiments, only the blood parameter sensor needs to be arranged at the flexible patch. In addition, only a corresponding functional module needs to be added to a main chip of the mobile terminal without adding corresponding hardware to the mobile terminal, which is easy to be implemented and can be applied to any mobile terminal having a wireless communication function.

In the following, a blood parameter monitoring method according to embodiments of the present disclosure is described.

A flowchart of a blood parameter monitoring method according to some embodiments of the present disclosure is shown in FIG. 5. The blood parameter monitoring method is applied in a blood parameter monitoring device. The blood parameter monitoring device includes a skin patch, a blood parameter sensor arranged at the skin patch, a processor connected to the blood parameter sensor, a timer connected to the processor and an alarm connected to the timer. The blood parameter monitoring method includes steps S501 to S504.

In step S501, the skin patch is worn on skin of an organism.

In step S502, a blood parameter of the organism is detected by the blood parameter sensor.

In step S503, it is determined by the processor whether the blood parameter detected by the blood parameter sensor is within a predetermined range; the processor sends an abnormality indication signal if the blood parameter is not within the predetermined range, and sends a normality indication signal if the blood parameter is within the predetermined range.

In step S504, the timer starts a timing process upon receiving the abnormality indication signal, stops the timing process upon receiving the normality indication signal, and sends an alarm signal if a duration of the timing process is longer than or equal to a predetermined duration.

In some embodiments of the present disclosure, the blood parameter monitoring device further includes a predetermined mobile communication terminal. And the blood parameter monitoring method further includes that, the alarm triggers, after receiving the alarm signal, the predetermined mobile communication terminal to send a warning signal to a preset phone number.

What described above includes preferable embodiments of the present disclosure. It should be noted that, various improvements and modifications can be made to the present disclosure by those skilled in the art without departing from the principle of the present disclosure, and these improvements and modifications shall fall within the scope of protection of the present disclosure.

1. A blood parameter monitoring device, comprising:
a skin patch, which is wearable on skin of an organism;
a blood parameter sensor, which is arranged at the skin patch and configured to detect a blood parameter of the organism;
a processor, which is connected to the blood parameter sensor and configured to send an abnormality indication signal in case of determining that the blood parameter detected by the blood parameter sensor is not within a predetermined range and send a normality indication signal in case of determining that the blood parameter detected by the blood parameter sensor is within the predetermined range;
a timer, which is connected to the processor and configured to start a timing process upon receiving the abnormality indication signal, stop the timing process upon receiving the normality indication signal, and send an alarm signal in a case that a duration of the timing process is longer than or equal to a predetermined duration; and
an alarm, which is connected to the timer and configured to give an alarm after receiving the alarm signal.

2. The blood parameter monitoring device according to claim 1, further comprising a predetermined mobile communication terminal, wherein the alarm is further configured to trigger, after receiving the alarm signal, the predetermined mobile communication terminal to send a warning signal to a preset phone number.

3. The blood parameter monitoring device according to claim 2, wherein the alarm comprises a transmitting element configured to transmit triggering information to the predetermined mobile communication terminal after the alarm signal is received, to trigger the predetermined mobile communication terminal to dial the preset phone number and/or send a warning message to the preset phone number.

4. The blood parameter monitoring device according to claim 1, wherein the blood parameter sensor comprises at least one of a blood flow sensor, a blood pressure sensor, a blood vessel resistance detector and a blood oxygen sensor.

5. The blood parameter monitoring device according to claim 1, wherein the processor, the timer and the alarm are all arranged at the skin patch.

6. The blood parameter monitoring device according to claim 2, wherein the processor, the timer and the alarm are all integrated in the predetermined mobile communication terminal;
the blood parameter sensor comprises: a first communication element arranged at the skin patch and configured to transmit the blood parameter to the processor;
a second communication element which is communicatively connected to the first communication element is arranged in the predetermined mobile communication terminal; and
the predetermined mobile communication terminal receives the blood parameter and transfers the blood parameter to the processor, via the second communication element.

7. The blood parameter monitoring device according to claim 6, wherein the predetermined mobile communication terminal is a phone, a computer or other device having a communication function, the first communication element and the second communication element are Bluetooth communication elements, infrared communication elements or other wireless communication elements.

8. The blood parameter monitoring device according to claim 1, wherein the skin patch is a flexible patch.

9. The blood parameter monitoring device according to claim 8, wherein the flexible patch is made of a plastic or a rubber and has a strip shape;
the flexible patch having the strip shape comprises one end provided with at least one circular hole and another end provided with a convex matching each of the at least one circular hole; or
the flexible patch having the strip shape has two ends each provided with an adhesive portion.

10. The blood parameter monitoring device according to claim 8, wherein the flexible patch is made of a synthetic fiber or a natural fiber; and
the flexible patch is provided with a fixing portion that enables the flexible patch to be wearable on the skin of the organism.

11. The blood parameter monitoring device according to claim 10, wherein,
the flexible patch comprises an electro-active fabric; and
the blood parameter sensor and the flexible patch are integral.

12. The blood parameter monitoring device according to claim 11, wherein in a case that the processor, the timer and the alarm are all arranged at the skin patch, the blood parameter sensor further comprises a power source configured to provide electric energy to the processor, the timer and the alarm; and
the power source is directly integrated in the flexible patch.

13. A blood parameter monitoring method, applied in a blood parameter monitoring device comprising a skin patch, a blood parameter sensor arranged at the skin patch, a processor connected to the blood parameter sensor, a timer connected to the processor and an alarm connected to the timer, wherein the blood parameter monitoring method comprises:
wearing the skin patch on skin of an organism;
detecting, by the blood parameter sensor, a blood parameter of the organism;
determining, by the processor, whether the blood parameter detected by the blood parameter sensor is within a predetermined range, sending an abnormality indication signal in a case that the blood parameter is not within the predetermined range, and sending a normality indication signal in a case that the blood parameter is within the predetermined range; and
starting, by the timer, a timing process upon receiving the abnormality indication signal, stopping, by the timer, the timing process upon receiving the normality indication signal, and sending, by the timer, an alarm signal in a case that a duration of the timing process is longer than or equal to a predetermined duration.

14. The blood parameter monitoring method according to claim 13, wherein the blood parameter monitoring device further comprises a predetermined mobile communication terminal, and the blood parameter monitoring method further comprises:
triggering, by the alarm after receiving the alarm signal, the predetermined mobile communication terminal to send a warning signal to a preset phone number.

15. The blood parameter monitoring device according to claim 2, wherein the blood parameter sensor comprises at least one of a blood flow sensor, a blood pressure sensor, a blood vessel resistance detector and a blood oxygen sensor.
